(19) United States
(12) Patent Application Publication
(10) Pub. No.: US 2002/0120215 A1
(43) Pub. Date: Aug. 29, 2002

(54) Blood collection set with retractable needle

(76) Inventors: Jamieson William Maclean Crawford, New York, NY (US); Stefanie Livanos, Bethlehem, PA (US)

Correspondence Address: BECTON, DICKINSON AND COMPANY, 1 BECTON DRIVE, FRANKLIN LAKES, NJ, 07417-1880, US

(21) Appl. No.: 10/067,107

(22) Filed: Nov. 13, 2001

Related U.S. Application Data
(60) Provisional application No. 60/260,032, filed on Jan. 5, 2001.

(51) Int. Cl. A61B 5/00

(52) U.S. Cl. 600/573

(57) ABSTRACT

An automatically shieldable blood collection set is provided. The blood collection set includes a needle assembly having a hub to which a needle cannula is fixedly attached. A safety shield is telescoped relative to the hub and the needle cannula such that the hub and the needle cannula can be moved from a distal position where the needle cannula is exposed to a proximal position where the needle cannula is safely shielded. A spring is provided between the shield and the hub to propel the hub and needle cannula proximally relative to the shield and into surrounding relationship with the needle cannula. A retainer is provided for releasably holding the hub and needle cannula in a distal ready-to-use condition relative to the shield. An actuator releases the retainer and enables the hub and needle cannula to be propelled by the spring. A lock may be provided for preventing inadvertent re-exposure of the needle cannula.

### BACKGROUND OF THE INVENTION

1. Field of the Invention

The subject invention relates to a blood collection set having a needle cannula that can be retracted into a shield to safely shield the needle cannula.

2. Description of the Related Art

Prior art blood collection set includes a small diameter needle cannula having a pointed distal end and a proximal end mounted to a thermoplastic hub. Portions of the blood collection set near the hub may be provided with a pair of flexible wings. The wings can be folded into face-to-face engagement with one another to facilitate digital manipulation of the small needle cannula. The wings then can be folded away from one another and taped into face-to-face engagement with the skin of the patient near a puncture site, The prior art blood collection set further includes a flexible plastic tube that has one end connected to the hub and an opposed end connected to a fitting. The fitting can be placed in communication with a reservoir to which collected blood may be directed.

The needle cannula of the prior art blood collection set typically is shielded prior to and after use to prevent accidental sticks. Needle shields used with prior art blood collection sets have taken many forms. Typically, a prior art blood collection set is packaged with a rigid tubular cap telescoped over the needle cannula to prevent accidental sticks prior to use. This tubular cap is removed from the needle cannula immediately prior to use of the blood collection set. Most prior art blood collection sets further include a second shield that is telescoped over the needle cannula and hub. The second shield may include at least one slot through which wings of the prior art hub may extend. Thus, the medical technician who uses the prior art blood collection set will hold the wings of the needle hub in one hand and the shield in the other hand after removing the needle cannula from the patient or blood donor. The wings then are slid proximally relative to the shield, thereby drawing the needle cannula into the shield. Some prior art shields are configured to engage the wings when the needle cannula has been shielded to make a re-exposure of the needle cannula difficult.

The digital manipulation that is required to shield the used needle cannula of a prior art blood collection set creates the potential for generating the accidental needle stick that the shield is intended to avoid. In particular, it is undesirable to rely upon a shielding that requires two hands to be moved in opposite directions in proximity to the point of a used needle cannula. Accordingly, the inventors herein have recognized the desirability of providing an automatically shieldable needle cannula for a blood collection set.

### SUMMARY OF THE INVENTION

The subject invention relates to a blood collection set which comprises a needle cannula having a proximal end, a pointed distal end and a lumen extending therebetween. The blood collection set further includes a hub that may be molded from a thermoplastic material. The hub includes a proximal end, a distal end and a passage extending continuously therebetween. The distal end of the hub is securely mounted to the proximal end of the needle cannula. Thus the lumen through the needle cannula communicates with the passage through the hub.

The blood collection set may further include a length of flexible tubing having opposed proximal and distal ends. The distal end of the flexible tubing may be connected to the proximal end of the hub such that the lumen through the needle cannula and the passage through the hub both communicate with the passage through the flexible tubing. The flexible tubing further includes a proximal end that may be connected to a fitting. The fitting may comprise a needle cannula that enables the blood collection set to be placed in communication with a reservoir for receiving a sample of blood. The tubing and the fitting may be of conventional design.

The blood collection set may further include a substantially rigid generally tubular safety cap mounted over the needle cannula for protection against accidental needle sticks prior to use of the blood collection set. The safety cap may include a proximal end that is frictionally engaged with the hub. The rigid tubular safety cap may be removed immediately prior to use of the blood collection set.

The blood collection set further includes a safety shield that is telescoped over a portion of the hub. The safety shield and the hub are dimensioned to permit relative movement therebetween. More particularly, the combined hub and needle cannula can be moved from a distal position where the needle cannula is exposed for use and a proximal position where the needle cannula is within the safety shield. Biasing means are provided between the safety shield and the hub for urging the hub proximally relative to the safety shield. The biasing means may be a coil spring that surrounds a portion of the hub.

The blood collection set further comprises a retainer for releasably retaining the hub and needle cannula in the distal position relative to the safety shield and against the stored energy of the biasing means. Actuation of the retainer causes the biasing means to retract the hub and the needle cannula into the safety shield.

### DESCRIPTION OF THE DRAWINGS

FIG. 1 is a perspective view of a blood collection set in accordance with the subject invention.

FIG. 2 is a cross-sectional view taken along line 2-2 in FIG. 1.

FIG. 3 is a perspective view similar to FIG. 1, but showing the shield protectively locked around the needle cannula.

### DETAILED DESCRIPTION

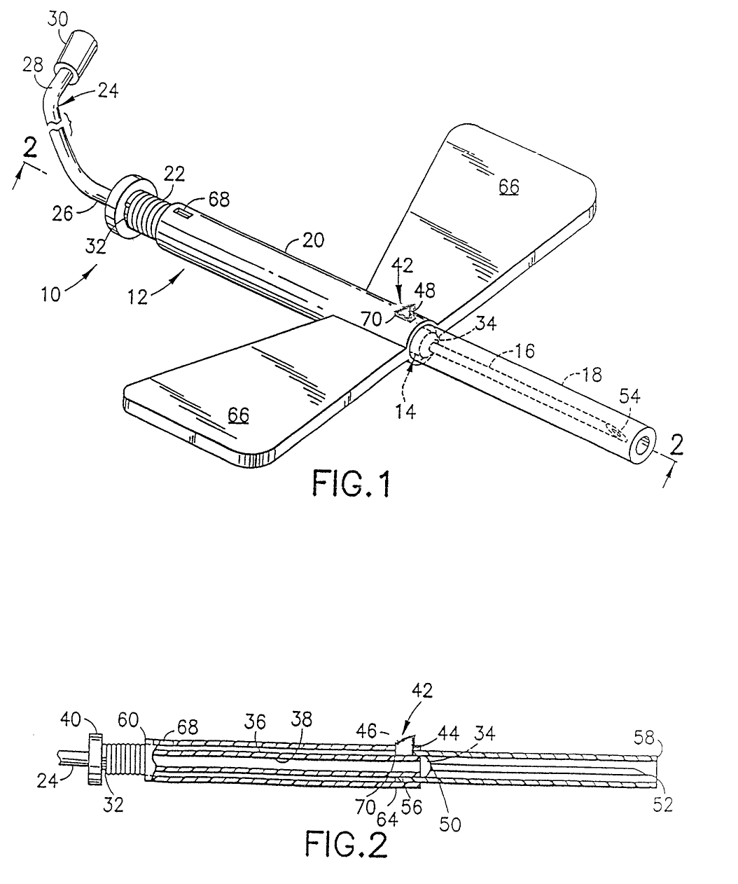

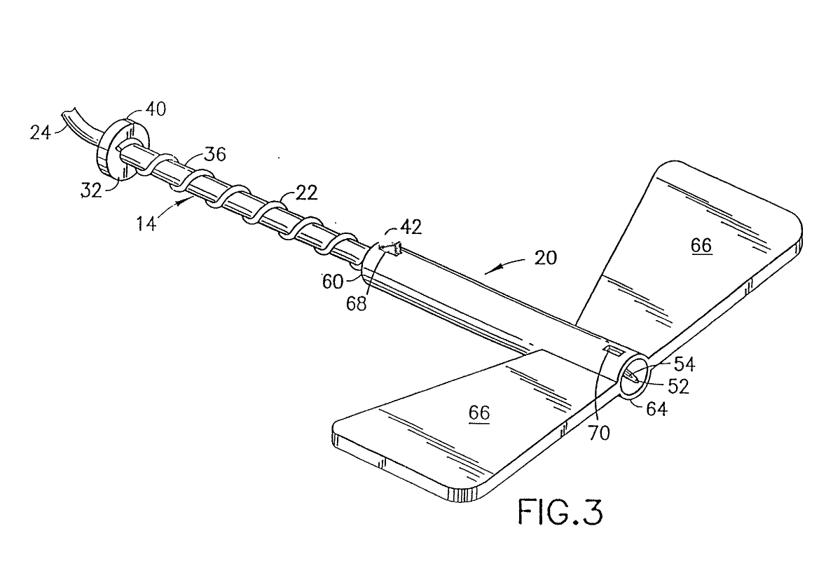

A blood collection set in accordance with the subject invention is identified generally by the numeral 10 in FIGS. 1-3. Blood collection set 10 includes a needle assembly 12 having a hub 14, a needle cannula 16, a safety cap 18, a safety shield 20 and a spring 22. Blood collection set 10 further includes a flexible tube 24 having a distal end 26 connected to needle assembly 12 and a proximal end 28 connected to a fitting 30. Fitting 30 is configured to be placed in communication with a reservoir into which blood drawn by needle assembly 12 may be deposited. Flexible tube 24 and fitting 30 may be of conventional prior art construction.

Hub 14 is formed from a thermoplastic material and includes a proximal end 32, a distal end 34 and a rigid tube 36 that extends axially between the ends. Tube 36 defines a passage 38 extending axially therethrough. An annular flange 40 is mounted on proximal end 32 of hub 14 and defines an outside diameter greater than the outside diameter of rigid tube 36 of hub 14.

Hub 14 further includes an actuator 42 that projects radially outwardly from a location on hub 14 near distal end 34. Actuator 42 defines a distal face 44 which, as explained further herein, functions as a retainer for releasably retaining shield 20 in a retracted position. Actuator 42 further includes a proximal face 46 which extends from outer surface 40 by a radial dimension that is less than the radial dimension of distal face 44. A ramp face 48 extends angularly between distal face 44 and proximal face 46 of actuator 42.

Needle cannula 16 includes a proximal end 50, a pointed distal end 52 and a lumen 54 extending therebetween. Proximal end 50 is mounted securely in distal end 34 of hub 14, such that lumen 54 communicates with passage 38 through hub 14 and with flexible tube 24.

Safety cap 18 is a generally tubular structure that is unitarily molded from a rigid plastic material. Safety cap 18 has a proximal end 56 that is frictionally engaged at distal end 34 of hub 14 and a distal end 58 that extends distally beyond needle cannula 16 for preventing accidental sticks prior to use of blood collection set 10.

Safety shield 20 is a substantially cylindrical tube having a proximal end 60 and a distal end 64. A pair of flexible wings 66 project transversely from a location on safety shield 20 near distal end 64. As shown in FIG. 1, wings 66 are substantially coplanar and enable needle assembly 12 to be taped to the skin of a patient in proximity to a puncture site. However, wings 66 can be folded approximately 90° toward one another to provide a convenient handle for gripping needle assembly 12. Such gripping is helpful prior to use of needle assembly 12 when safety cap 18 is being removed and when needle cannula 16 is being inserted into a patient.

Safety shield 20 defines an inside diameter which is less than the outside diameter of flange 40 on hub 14. However, the inside diameter of safety shield 20 is greater than the sum of the outside diameter of tube 36 of hub 14 plus the radial dimension of distal face 44 on actuator 42. Thus, shield 20 can be slid over actuator 42 on hub 14.

Safety shield 20 includes a proximal locking aperture 68 and a distal retaining aperture 70 each of which is dimensioned to receive actuator 42. Safety shield 20 defines a length measured from locking aperture 68 to distal end 64 that exceeds the distance from actuator 42 to distal end 52 of needle cannula 16.

Spring 22 surrounds rigid tube 36 of hub 14. Spring 22 has an inside diameter smaller than the outside diameter of flange 40 and an outside diameter smaller than the inside diameter of safety shield 20. Spring 22 is in a compressed state when hub 14 is in its distal position relative to safety shield 20, as shown in FIGS. 1 and 2.

Needle assembly 12 is used in a conventional manner by initially folding wings 66 toward one another to enable convenient digital manipulation of needle assembly 12. Safety cap 18 then is slidably removed from needle cannula 16, and needle cannula 16 is inserted into a patient. Wings 66 then may be folded flat against the skin of the patient and taped in place. Flexible tube 24 and fitting 30 will be used in conventional manner for connecting blood collection set 10 to a bag or other reservoir. Upon collection of a sufficient volume of blood, any tape that had held wings 66 to the skin of a patient will be removed, and needle assembly 12 then is withdrawn.

To prevent accidental sticks with the contaminated needle cannula 16, the medical an need merely squeeze actuator 42 and safety shield 20 between a thumb and forefinger. squeezing will urge the actuator 42 within the safety shield 20, and forces of spring 22 will b 14 and needle cannula 16 proximally. Spring 22 will continue to propel hub 14 and cannula 16 in a proximal direction and into shielding relationship within safety shield 20. After distal end 64 of safety shield 20 is distally beyond pointed distal end 52 of needle cannula 16, actuator 42 will align with locking aperture 68 in shield 20. The guiding of actuator 42 into locking aperture 68 can be achieved by a tapering of interior surface regions of safety shield 20 at loctions opposite locking aperture 68. In this condition, distal face 44 of actuator 42 will engage against distal portions of locking aperture 68 to prevent the re-exposure of needle cannula 16. Additionally, distal portions of locking aperture 68 will engage against distal face 44 of actuator 42 to prevent a complete removal of safety shield 20 from hub 14.

What is claimed is:

1. A blood collection set comprising a fitting for connection to a blood collection reservoir, a flexible tube extending from said fitting and a needle assembly connected to an end of the flexible tube remote from the fitting, said needle assembly comprising:
a hub having a proximal end connected to said flexible tube, a distal end and a passage extending therebetween for providing fluid communication with said flexible tube;
a needle cannula having a proximal end rigidly connected to said distal end of said hub, a pointed distal end and a lumen extending therebetween, said lumen providing fluid communication with said passage through said hub;
a shield telescoped with said hub and said needle cannula, such that said hub and said needle cannula are movable from a distal position where said needle cannula is exposed to a proximal position where said needle cannula is shielded;
a spring captured between portions of said hub and said shield and operative for propelling said hub and said needle cannula into said proximal position relative to said shield;
a retainer for releasably retaining the hub and said needle cannula in the distal position; and
an actuator for selectively releasing said retainer.

2. The blood collection set of claim 1, further comprising a lock for locking said hub and said needle cannula in said proximal position.

3. The blood collection set of claim 1, wherein said shield is substantially tubular, and wherein said blood collection set further comprises a pair of flexible wings projecting transversely from said shield.

4. The blood collection set of claim 3, wherein the shield includes opposed proximal and distal ends, the wings projecting transversely from the shield at a location substantially adjacent the distal end of the shield.

5. The blood collection set of claim 1, wherein the shield is substantially tubular with opposed proximal and distal ends, a distal aperture extending through the shield at a location in proximity to the distal end, a projection extending outwardly from the needle hub and passing into the distal aperture adjacent the shield when the hub and the needle cannula are in the distal position, the retainer defining a first surface on the projection disposed for engaging the shield adjacent the distal aperture in the shield, the actuator defining a second surface on the projection extending outwardly from the shield and being depressible sufficiently for disengaging said retainer from said portions of said shield adjacent said aperture.

6. The blood collection set of claim 5, wherein the projection further comprises a ramp face extending from the actuator surface on the projection to the retainer surface on the projection, the ramp face being aligned at an acute angle to the needle cannula.

7. The blood collection set of claim 5, wherein the shield further comprises a proximal aperture extending therethrough at a location substantially adjacent the proximal end of the shield, the projection being dimensioned for locked engagement in the proximal aperture for preventing distal movement of the hub and the needle cannula relative to the shield.

8. A needle assembly comprising:
a hub having a proximal end, distal end and a passage extending therebetween for providing fluid communication through said hub;
a needle cannula mounted to the distal end of the hub;
a shield telescoped with said hub and said needle cannula, such that said hub and said needle cannula are movable from a distal position where said needle cannula is exposed to a proximal position where said needle cannula is enclosed within said shield;
a spring captured between portions of said hub and said shield and being operative for propelling said hub and said needle cannula into said proximal position relative to said shield;
a retainer for releasably retaining the hub and said needle cannula in the distal position; and
an actuator for selectively releasing said retainer.

9. The needle assembly of claim 8, further comprising a lock for locking said hub and said needle cannula in said proximal position.

10. The needle assembly of claim 8, wherein said shield is substantially tubular, and wherein said blood collection set further comprises a pair of flexible wings projecting transversely from said shield.

11. The needle assembly of claim 10, wherein the shield includes opposed proximal and distal ends, the wings projecting transversely from the shield at a location substantially adjacent the distal end of the shield.

12. The needle assembly of claim 8, wherein the shield is substantially tubular with opposed proximal and distal ends, a distal aperture extending through the shield in proximity to the distal end, a projection extending outwardly from the needle hub and passing into the distal aperture of the shield when the hub and the needle cannula are in the distal position, the retainer defining a first surface on the projection disposed for engaging the shield adjacent the distal aperture in the shield, the actuator defining a second surface on the projection extending outwardly from the shield and being depressible sufficiently for disengaging said retainer from said portions of said shield adjacent said aperture.

13. The needle assembly of claim 12, wherein the projection further comprises a ramp face extending from the actuator surface on the projection to the retainer surface on the projection, the ramp face being aligned at an acute angle to the needle cannula.

14. The needle assembly of claim 12, wherein the shield further comprises a proximal aperture extending therethrough at a location substantially adjacent the proximal end of the shield, the projection being dimensioned for locked engagement in the proximal aperture for preventing distal movement of the hub and the needle cannula relative to the shield.
